(19) United States
(12) Patent Application Publication
(10) Pub. No.: US 2005/0107820 A1
(43) Pub. Date: May 19, 2005

(54) Vascular puncture depth locator

(76) Inventors: Andrew Thomas Forsberg, Minneapolis, MN (US); Loran Paprocki, St. Louis Park, MN (US)

Correspondence Address: L. Grant Foster;HOLLAND & HART LLP, 555 - 17th Street, Suite 3200, P.O. Box 8749, Denver, CO, 80201, US

(21) Appl. No.: 10/713,530

(22) Filed: Nov. 13, 2003

(51) Int. Cl. A61M 29/00

(52) U.S. Cl. 606/194

(57) ABSTRACT

The present invention provides dilator and insertion sheath assembly having penetration locators. In particular, at least one inlet port resides on the insertion sheath and is in fluid communication with a drip hole to provide penetration indication to a user.

### FIELD OF THE INVENTION

The present invention relates to a vascular sheath and dilator and, more particularly, to a vascular sheath and dilator having at least one puncture depth locator in the sheath.

### BACKGROUND OF THE INVENTION

Vascular insertion of devices or arterial seals requires puncture of an arterial vessel and placement of the device or seal. FIGS. 1, 2 and 3 show a conventional insertion sheath 102 and dilator 202 useful for vascular penetration of a blood vessel 302 in a patient 304. Insertion sheath 102 has a sheath distal end 104 and a sheath proximate end 106. Sheath distal end 104 contains a tool access port 108, and sheath proximate end 106 has a tool junction port 108. Dilator 202 has a dilator distal end 204 and a dilator proximate end 206. Dilator distal end 204 contains an inlet port 208. Dilator proximate end 206 contains a tool junction connector 210 and a drip hole 212. Inlet port 208 is in fluid communication with drip hole 212 via a lumen (not specifically shown in FIGS. 1 and 2) contained in dilator 202.

Placing a device, such as, a vascular seal, using insertion sheath 102 and dilator 202 will now be explained. First, a puncture site 306 is located that will allow access to blood vessel 302, such as the femoral artery. Dilator 202 is placed in insertion sheath 102 so that sheath distal end 104 is substantially adjacent dilator distal end 204 and tool junction port 108 and tool junction connector 210 mate. Dilator 202 is somewhat longer than insertion sheath 102 such that inlet port 208 resides outside sheath distal end 104 a predetermined distance d.

Using conventional techniques, dilator 202 and insertion sheath 102 are inserted through puncture site 306 into blood vessel 302 of patient 304. As sheath distal end 104 penetrates blood vessel 302, blood will flow from inlet port 208 to drip hole 212 via the dilator lumen (not shown).

Blood exiting drip hole 212 indicates insertion sheath 102 has just penetrated blood vessel 302. To ensure proper placement of structure, insertion sheath 102 and dilator 202 are backed out of the vessel until blood stops flowing from drip hole 212. Next, insertion sheath 102 and dilator 202 are re-inserted in blood vessel 302 until blood starts flowing from drip hole 212. Proper depth of penetration and location of the assembly is established by continuing to insert and additional distance, for example, a doctor would insert the assembly 1 to 2 centimeters for the femoral artery. Once properly installed, the vascular seal or vascular tool can be inserted via insertion sheath 102 and introduced to blood vessel 302.

The above assembly has various drawbacks, such as, for example, over inserting the assembly. Also, because inlet port 208 is located on dilator distal end 204 it is at best only a proximate location indicator for the location of sheath distal end 104. Thus, it would be desirous to provide an improved vascular penetration depth locator.

### SUMMARY OF THE INVENTION

To attain the advantages and in accordance with the purpose of the invention, as embodied and broadly described herein, a vascular insertion assembly is provided. The vascular insertion assembly includes an insertion sheath and a dilator. The dilator is designed to fit snuggly in the insertion sheath. A first inlet port is located towards the sheath distal end and a first indicator is located towards the proximate end. The first indicator provides penetration indication when the first inlet port penetrates the vessel.

The foregoing and other features, utilities and advantages of the invention will be apparent from the following more particular description of a preferred embodiment of the invention as illustrated in the accompanying drawings.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is a perspective view of a conventional insertion sheath and dilator;

FIG. 2 is a perspective view of a conventional insertion sheath and dilator;

FIG. 3 is a perspective view of a conventional insertion sheath and dilator in use;

FIG. 4 is a perspective view of an assembly consistent with the present invention;

FIG. 5 is a perspective view of a portion of an assembly consistent with the present invention;

FIG. 6 is a perspective view of a portion of an assembly consistent with the present invention;

FIG. 7 is a cross sectional view of a gauge usable with an assembly consistent with the present invention;

FIG. 8 is a front plan view of a faceplate of a gauge usable with an assembly consistent with the present invention; and

FIG. 9A to 9C shows use of an assembly consistent with the present invention.

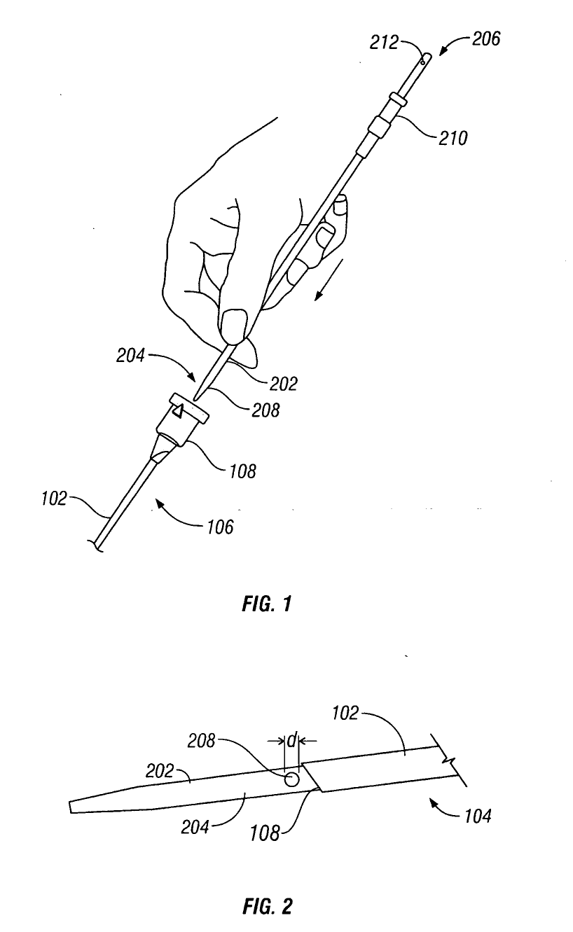

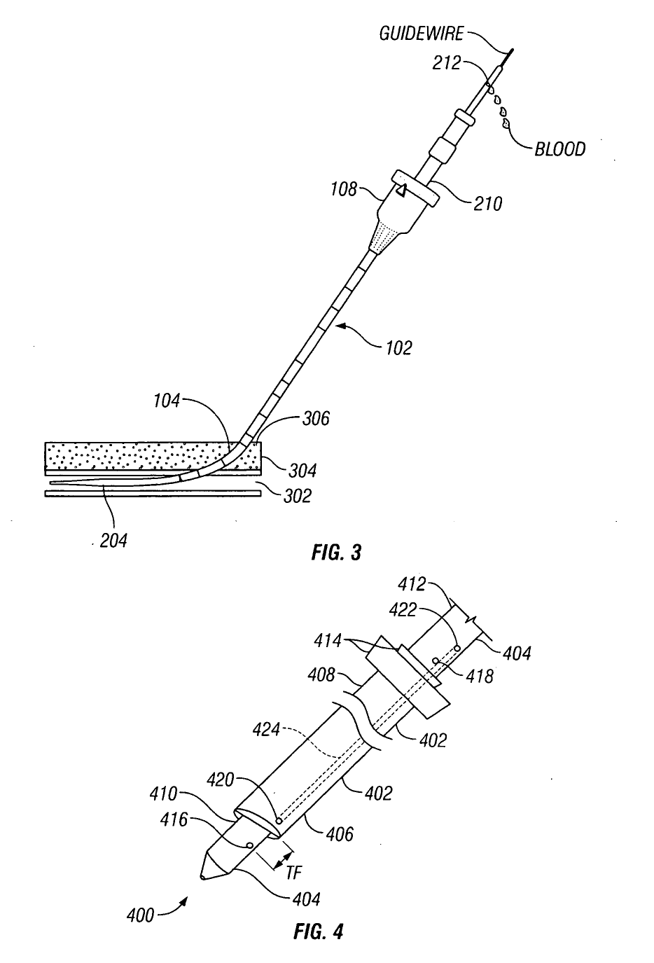

### DETAILED DESCRIPTION

The present invention will now be described with reference to FIGS. 4-9C. Referring to FIG. 4, an assembly 400 consistent with the present invention is shown. Assembly 400 includes an insertion sheath 402 and a dilator 404. Insertion sheath 402 includes a sheath distal end 406 and a sheath proximate end 408. Dilator 404 includes a dilator distal end 410 and a dilator proximate end 412. Insertion sheath 402 and dilator 404 are coupled using a conventional mating 414. Similar to conventional assemblies (described above), dilator distal end 410 contains an inlet port 416, and dilator proximate end 412 contains a drip hole 418. Inlet port 416 and drip hole 418 are in fluid communication via a first lumen (not shown) in dilator 404 or the like. Unlike conventional assemblies, however, assembly 400 contains a over insertion indication port 420 located in sheath distal end 406, and over insertion drip hole 422 located in dilator proximate end 412 (as shown). Over insertion indication port 420 and over insertion drip hole 422 are in fluid communication via a second lumen 424 (shown in phantom) that exists separate from the first lumen. Inlet port 416 and over insertion indication port 420 can be located a predetermined distance TF to indicate assembly 400 has been inserted too far. While drip hole 418 and over insertion drip hole 422 are shown arranged sequentially for when blood would flow, alternative arrangements are possible. Also, while over insertion drip hole 422 is shown arranged on dilator proximate end 412, over insertion drip hole 422 could be arranged on sheath proximate end 408, which may actually facilitate manufacturing of assembly 400.

In use, assembly 400 would first be inserted until vessel penetration was indicated, as with the prior art device above. Using the prior art device, care must be exercised during reinsertion of the assembly approximately 1 to 2 centimeters to ensure it is not over inserted. Using assembly 400, however, over insertion indicator port 420 would provide over insertion indication when blood begins flowing from over insertion drip hole 422. Thus, assisting proper location of assembly 400 and avoiding over insertion. Distance TF could be selected to provide indication that assembly 400 is about to be over inserted (i.e., blood flows from over insertion drip hole 422 prior to over insertion) or to provide indication that over insertion just occurred (i.e., blood flows from over insertion drip hole 422 at or just after over insertion).

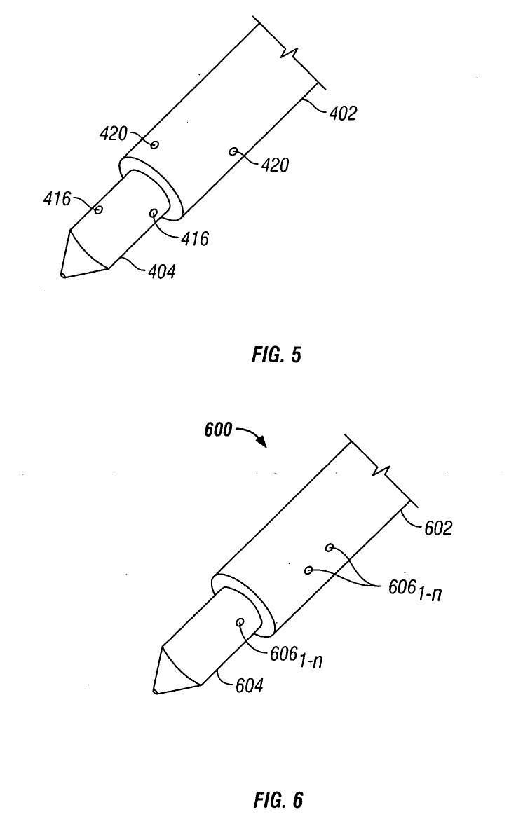

Assembly 400 is shown with inlet port 416 and drip hole 418 as the primary artery entry sensor holes. Over insertion indication port 420 and over insertion drip hole 422 are the insertion too far sensor holes. Alternative arrangements for these sensor holes are possible. For example, referring to FIG. 5, multiple inlet ports 416 could be provided in dilator 404 and multiple over insertion indication ports 420 could be provided in insertion sheath 402 to provide better indication of entry and over insertion. Moreover, as shown, the ports could be offset or staggered to accommodate an insertion angle for assembly 400.

Fluid communication is provided between ports 416 and 420 and holes 418 and 422 by separate fluid conduits. It is believed separate lumens would work well, but other types of tubes or capillaries could be used. Moreover, a single lumen having multiple and separate flow paths would work as well. Lumens could be contained in the dilator or in the sheath as a matter of design choice. Having the lumen contained in the dilator would inhibit locating when the dilator is removed.

Referring now to FIG. 6, a lower portion 600 of an insertion sheath 602 and a dilator 604 is shown. In this case, multiple ports 606, n are shown. One of skill in the art would now recognize that corresponding drip holes and flow paths, such as, for example, lumens, would exists, but that they are not shown for ease of reference. In this case, the ports could provide indication of initial insertion, proper insertion, and over insertion. More or less ports and drip holes could be used to provide additional indications. For example, the port on dilator 604 could be removed and location information could be provided using only the proper insertion indication and over insertion indication. Still further, the over insertion indication could be removed leaving only the proper insertion port. As can be seen, the combination of ports is limited only by the design of the insertion sheath.

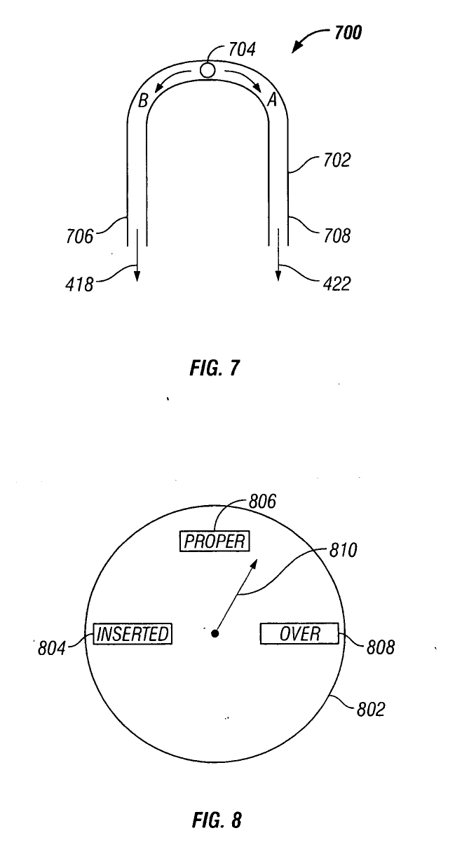

Referring to FIG. 4, location indication is provided by fluid flow out of drip hole 418 and/or over insertion drip hole 422. Alternative means of indication are possible, however. As shown by FIG. 7, a differential pressure gauge 700 could be supplied for indication. Differential pressure gauge 700, in this case, is a ball float gauge comprising a tube 702 and a suspended ball 704. Tube 702 has two access ports 706 and 708. Access port 706 could be connected to drip hole 418 and access port 708 could be connected to over insertion drip hole 422. In this case, when inlet port 416 entered a vessel, blood flow out of drip hole 418 would cause ball 704 to move towards access port 708 as shown by arrow A. If the assembly was inserted to far, blood flow out of over insertion drip hole 422 would cause ball 704 to move towards access port 706 as shown by arrow A. Thus, use of the gauge would provide visual indication without allowing blood to freely drip in the surgical arena. Alternatively, as shown by FIG. 8, differential pressure gauge 700 could have a more conventional looking faceplate 802. Faceplate 802 may provide various indications such as the point of initial insertion as shown by inserted indicator 804, proper insertion as shown by proper indicator 806, and over insertion as shown by over indicator 808. An arrow 810 or other pointer could be used to provide reference indication.

Finally, while the indicators are shown as largely mechanical in nature, electrical components could be used. For example, over insertion indication port 420 could be replaced with a fluid sensor or a pressure sensor. A wire could replace lumen 424. And a light emitting diode or the like could replace over insertion drip hole 424. Thus, when blood or some other fluid contacted the fluid sensor, an electrical signal would be sent via the wire to the LED that would light and provide over insertion indication. Alternatively, over insertion indication port 420 and lumen 424 could provide fluid communication to a fluid sensor replacing over insertion drip hole 422. In this case, when port 420 entered fluid, fluid communicated via lumen 424 would cause the fluid sensor to provide a visual indication, an audio indication, or a combination thereof.

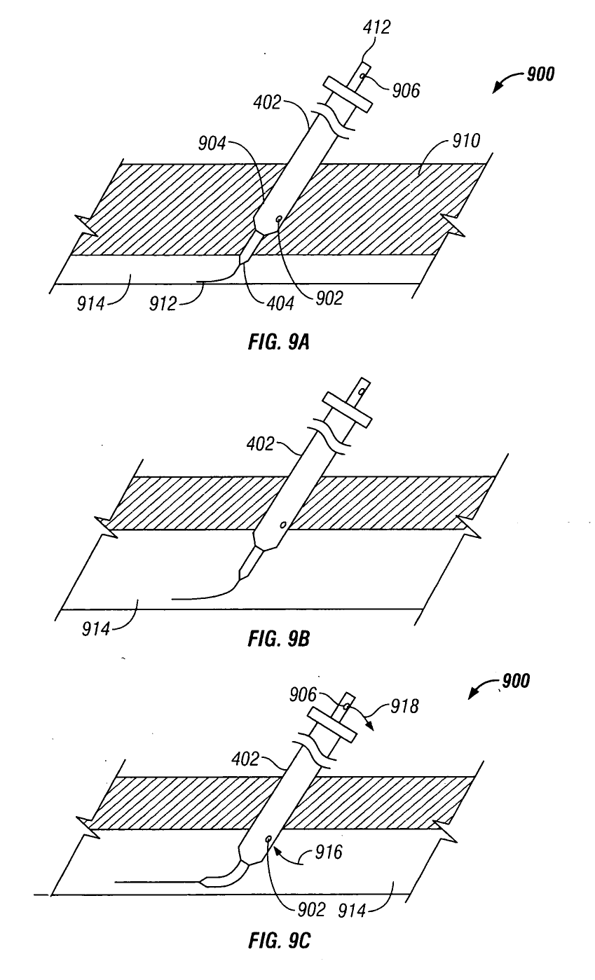

FIG. 9 shows use of an assembly 900 consistent with the present invention. In this example, assembly 900 contains dilator 404 and insertion sheath 402. Dilator 404 does not contain an inlet port and insertion sheath 402 contains proper insertion indication port 902 in a sheath distal end 904. A corresponding proper insertion drip hole 906 is located in a dilator proximate end 412. As shown, assembly 900 is inserted through a patient 910 over a guide wire 912. Dilator 404 punctures vessel 914 prior to insertion sheath 402 entering vessel 914, FIG. 9A. FIG. 9B shows insertion sheath 402 after it has entered vessel 914 but prior to achieving proper insertion depth. FIG. 9C shows insertion sheath 402 having achieved proper insertion depth. At this point, at least a portion of port 902 has entered vessel 914 and fluid flows from vessel 914 into port 902 as shown by arrow 916 and out drip hole 906 as shown by arrow 918, indicating proper insertion of insertion sheath 402. At this point, dilator 404 and guide wire 912 can be removed. If fluid communication between port 902 and drip hole 906 is provided by a lumen (not shown) in dilator 404, removal of dilator 404 removes fluid flow from drip hole 906. If the lumen (not shown) is provided in sheath 402, however, positive location information is communicated to the Doctor even after removal of dilator 404 and guide wire 912.

While the invention has been particularly shown and described with reference to embodiments thereof, it will be understood by those skilled in the art that various other changes in the form and details may be made without departing from the spirit and scope of the invention.

1. A vascular insertion assembly, comprising:
an insertion sheath;
a dilator;
the insertion sheath having an inside diameter and comprising a sheath distal end and a sheath proximate end;
the dilator sized to fit in the inside diameter of the insertion sheath comprising a dilator distal end and a dilator proximate end;
a first inlet port located about the sheath distal end;
a first indicator; and
the first indicator coupled with the first inlet port, such that when the first inlet port penetrates a vessel the first indicator provides indication.

2. The assembly according to claim 1, further comprising:
a second inlet port located about the dilator-distal end;
a second indicator; and
the second indicator coupled with the second inlet port, such that when the second inlet port penetrates a vessel the second indicator provides indication.

3. The assembly according to claim 1, wherein the first inlet port comprises a plurality of inlet ports.

4. The assembly according to claim 3, wherein the plurality of inlet ports are offset to accommodate an insertion angle of the assembly.

5. The assembly according to claim 2, further comprising:
at least a third inlet port;
at least a third indicator; and
at least the third indicator coupled with at least the third inlet port, such that when at least the third inlet port penetrates a vessel at least the third indicator provides indication.

6. The assembly according to claim 2, wherein the second inlet port and the second indicator provide indication of an initial penetration of the insertion sheath to the vessel.

7. The assembly according to claim 2, wherein the first inlet port and the first indicator provide indication of too far penetration of the insertion sheath to the vessel.

8. The assembly according to claim 2, wherein the first inlet port and the first indicator provide indication of proper penetration of the insertion sheath to the vessel.

9. The assembly according to claim 1, wherein the first inlet port and the first indicator provide indication of too far penetration of the insertion sheath to the vessel.

10. The assembly according to claim 1, wherein the first inlet port and the first indicator provide indication of proper penetration of the insertion sheath to the vessel.

11. The assembly according to claim 1, wherein the first inlet port and the first indicator provide indication of initial penetration of the insertion sheath to the vessel.

12. The assembly according to claim 5, wherein:
the second inlet port and the second indicator provide indication of an initial penetration of the insertion sheath to the vessel;
the first inlet port and the first indicator provide indication of proper penetration of the insertion sheath to the vessel; and
at least the third inlet port and at least the third indicator provide indication of too far penetration of the insertion sheath to the vessel.

13. The assembly according to claim 1, wherein the first indicator is a first drip hole in fluid communication with the first inlet port.

14. The assembly according to claim 13, further comprising:
a first lumen providing the fluid communication between the first drip hole and the first inlet port.

15. The assembly according to claim 13, wherein the first lumen is located in the dilator.

16. The assembly according to claim 13, wherein the first lumen is located in a wall of the insertion sheath.

17. The assembly according to claim 2, wherein the first indicator is a first drip hole in fluid communication with the first inlet port and the second indicator is a second drip hole in fluid communication with the second inlet port.

18. The assembly according to claim 17, further comprising:
a first lumen provides the fluid communication between the first drip hole and the first inlet port; and
a second lumen provides the fluid communication between the second drip hole and the second inlet port.

19. The assembly according to claim 18, wherein at least one of the first lumen and the second lumen is located in the dilator.

20. The assembly according to claim 18, wherein at least one of the first lumen and the second lumen is located in a wall of the insertion sheath.

21. The assembly according to claim 17, further comprising:
a lumen;
the lumen comprises a first flow path and a second flow path, wherein
the first flow path provides the fluid communication between the first drip hole and the first inlet port; and
a second flow path provides the fluid communication between the second drip hole and the second inlet port.

22. The assembly according to claim 1, wherein the first indicator is a gauge.

23. The assembly according to claim 22, wherein the gauge is a pressure gauge.

24. The assembly according to claim 22, further comprising:
a second inlet port located about the dilator distal end; wherein
the gauge is a differential pressure gauge and the first inlet port is coupled to a first input port of the differential pressure gauge and the second inlet port is coupled to a second input port of the differential pressure gauge, such that the differential pressure gauge indicates when the second inlet port and the first inlet port penetrate the vessel.

25. The assembly according to claim 24, wherein the differential pressure gauge is a ball, float gauge.

26. The assembly according to claim 24, wherein the differential pressure gauge contains a faceplate.

27. The assembly according to claim 26, wherein the faceplate provide penetration information.

28. A vascular insertion assembly, comprising:
an insertion sheath having a sheath distal end and a sheath proximate end;
a dilator having a dilator distal end and a dilator proximate end;
the dilator sized to fit in the insertion sheath;
a first inlet port located about the dilator distal end;
a second inlet port located about the sheath distal end;
a first drip hole in fluid communication with the first inlet port; and
a second drip hole in fluid communication with the second inlet port, wherein fluid flows out the first drip hole when the first inlet port penetrates a vessel and fluid flows out the second drip hole when the second inlet port penetrates the vessel.

29. The assembly according to claim 28, wherein fluid communication is provided via at least one lumen.

30. The assembly according to claim 28, wherein the first inlet port comprises a plurality of first inlet ports.

31. The assembly according to claim 30, wherein the plurality of first inlet ports are staggered.

32. The assembly according to claim 28, wherein the second inlet port comprises a plurality of second inlet ports.

33. The assembly according to claim 30, wherein the second inlet port comprises a plurality of second inlet ports.

34. The assembly according to claim 33, wherein at least one of the plurality of first inlet port and the plurality of second inlet ports are staggered.

35. The assembly according to claim 28, further comprising:
a penetration gauge;
the penetration gauge comprising an indicator, a first access port, and a second access port;
the first access port in fluid communication with the first drip hole and the second access port in fluid communication with the second drip hole, such that the indicator provides indication of when the first inlet port and the second inlet port penetrate the vessel.

36. The assembly according to claim 28, further comprising:
a penetration gauge;
the penetration gauge comprising an indicator, a first fluid sensor, and a second fluid sensor;
the first fluid sensor adapted to generate a first signal when the first inlet port penetrates the vessel and the second fluid sensor adapted to generate a second signal when the second inlet port penetrates the vessel.

37. The assembly according to claim 28, wherein the first inlet port and first drip hole provide initial penetration indication of the vessel.

38. The assembly according to claim 28, wherein the second inlet port and second drip hole provide too far penetration indication of the vessel.

39. The assembly according to claim 28, further comprising:
at least a third inlet port; and
at least a third drip hole, such that at least the third inlet port and at least the third drip hole provide additional penetration information.

40. A vascular insertion assembly, comprising:
an insertion sheath having a sheath distal end and a sheath proximate end;
a dilator having a dilator distal end and a dilator proximate end;
the insertion sheath having an inside diameter the dilator having an outside diameter such that the dilator fits snuggly inside the insertion sheath; and
means for providing penetration information located about the insertion sheath.

41. The assembly according to claim 40, wherein the means for providing penetration information comprises at least a first inlet port in fluid communication with at least a first drip hole.

42. The assembly according to claim 41, wherein fluid communication is provided by at least one lumen residing in a wall of the insertion sheath.

43. The assembly according to claim 40, wherein the means for providing penetration information comprises at least a first fluid sensor electronically coupled to at least a first indicator.

44. The assembly according to claim 40, wherein the means for providing penetration information comprises at least a first inlet port in the dilator distal end and a second inlet port in the sheath distal end.
